(19) United States
(12) Patent Application Publication
(10) Pub. No.: US 2018/0325972 A1
(43) Pub. Date: Nov. 15, 2018

(54) CANNABIS-HEMP BEVERAGE INFUSIONS

(71) Applicant: David Harold Moore, Henderson, NV (US)

(72) Inventor: David Harold Moore, Henderson, NV (US)

(21) Appl. No.: 15/591,430

(22) Filed: May 10, 2017

(51) Int. Cl. A61K 36/185; A23L 5/10; A23L 2/72; A23L 2/52; A23L 3/36; A23L 2/38; A61K 9/00

(52) U.S. Cl. CPC A61K 36/185; A23L 5/10; A23L 2/72; A23L 2/52; A23L 3/36; A61K 2236/17; A61K 9/0095; A23V 2002/00; A61K 2236/37; A61K 2236/53; A61K 2236/15; A23L 2/382

(57) ABSTRACT

The present invention provides a commercial process for preparing the ‘Cannabis-Hemp Beverage Infusions’ into a soothing, impactful and pleasant beverage; that also acts as an enhanced recreational experience. The infusion process utilizes both a raw and twice-heat cooked ‘fusionable beverage’ blend that can be mixed with various fillers, herbs, and flavors; and poured into a drinking vessel of any type, or processed into a sealed drinking container for later use.

The commercial process entails the combining of various raw cannabinoids, decarboxylated cannabinoids, terpenes, terpenoids, and flavonoids that are found in the Cannabis-Hemp Plant; with other herbs, fillers, and flavors for the purposes of creating a pleasant and relaxing beverage that enhances our recreational experiences in a wholesome way.

To accomplish this process, the Cannabis-Hemp Plant must be correctly processed, and a quantity of the Cannabis-Hemp Plant must be correctly processed and twice-heat-cooked, with added flavorings. The heat-cooked Cannabis-Hemp plant may then be twice-heat cooked and blended with other herbs, flavorings, and chemicals for maximum efficacy.

### BACKGROUND

My name is David Harold Moore and am the inventor of the ‘Cannabis-Hemp Fusionable Beverage’ process. I have suffered from a High Functioning form of Autism, known as Asperger's Syndrome, since I was born. The autism caused the dysregulation of my endocannabinoid immune system, which caused the autoimmune disorder known as Celiac Disease, which then turned into cancer of the stomach lining, And only after going into renal failure, did I begin to look for a solution.

Then read a recent study from the Biomedical Center for Autism Research and Treatment, which suggests the CB2 receptor as a potential therapeutic target for the cannabis-hemp infused coconut-oil. This study was the first to demonstrate the up-regulation of CB2 receptors in autistic endocannabinoid systems.

The CB2 receptor enhancements demonstrate that the endocannabinoid system is involved in the maintenance of autism. According to the findings, the endocannabinoid system may represent a novel treatment opportunity for cannabis therapy with autistic minds. And that's because the up-regulation of the CB2 receptors enhances the peripheral organs ability to manage the immune system. And endocannabinoid signaling, in general, affects learning, pain, and other important regenerative processes.

You see, the endocannabinoid system is an intricate network of endocannabinoids, their receptors, and the associated enzymes; and all play critical roles in maintaining the body's homeostasis. And our biochemical homeostasis is unique to each one of us. It is therefore important to note, that in most cases, scientists make decisions based on evidence gained from an experiment with extensive controls. But the differences in cannabinoid composition, varies from batch to batch, thus making cannabis-hemp experiments too organic for scientific rigidity.

So I needed to find a healthy and inexpensive way to make a nutritional Cannabis-Hemp food grade dietary supplement. A daily dietary supplement that was all natural and healthy, a capsule with little to no psychoactivity (microdosing) and a capsule that could he taken in large quantities without harmful side effects. But there were no commercial products that would meet my needs.

Then I began experimenting with different ingredients and different heating methods. This process took about two years before I came up with a novel, non-obvious and useful method to process the Cannabis-Hemp plant into a food grade dietary supplement.

And this novel, non-obvious and useful method is a process based upon the different infusions and pressings of the raw and cooked cannabis-hemp plant, combined with various fats, to produce different amalgams that holistically synergize to create the wholesome food product, and nutritional dietary supplement, called ‘Cannabis-Hemp Paste’ (Ser. No. 14/969,497).

So after applying for the ‘Cannabis Hemp Paste Patent’, I started researching more about the endocannabinoid system, and found out that all of the animals on this planet also have an endocannabinoid system. Then I had another ‘crystallization moment’, where I realized that the cannabis-hemp plant must be decarboxylated by using my previously discovered methods, so our livestock could also realize the efficacy of the cannabis-hemp plant.

I then delved into animal suffering, animal feed, and animal antibiotics; and realized that the ‘Cannabis-Hemp Animal Feed’, if processed correctly, could reduce animal suffering, could reduce the need for animal antibiotics, and could reduce the need to cultivate so much of our arable farmland, to feed our livestock with.

Following the patent application for the ‘Cannabis-Hemp Animal Feed’ (Ser. No. 15/080,526), I started focusing on how to use the Cannabis-Hemp Plant for addiction recovery. And I began to read about how the Cannabis-Hemp Plant can help people with Heroin Addiction.

So I then extrapolated that understanding with the idea that the Cannabis-Hemp Plant could help people relieve their Tobacco Smoking addiction, and their Nicotine Chemical addiction. So I then came up with a blended recipe that could stand as a substitute for Tobacco Cigarettes, while at the same time, weaning the user off-of their Nicotine addiction.

I then started researching various herbs, flavorings, and chemicals that would help create a smooth and impactful Tobacco Replacement. And here is where I found out that the average Tobacco Cigarettes is filled with a majority of chemicals and fillers. In fact, the average Tobacco Cigarette has approximately 600 ingredients. And when burned, they create more than 7,000 chemicals. At least 69 of these chemicals are known to cause cancer, and many are poisonous.

Then I started to see why my Grandfather died of Lung Cancer, and I started to see why my friends could never quit smoking the chemically enhanced Tobacco Smoking Products. Then I started to realize, that for this deadly process to cease, we had to come up with a way for people to satisfy their cravings for the effects and rituals acquired during their Tobacco Smoking addiction. Meaning, we still had to provide the ritual of smoking, and we had to add various ingredients to enhance, smooth-out, and magnify the smoking experience, while also remembering to pre-decarboxylate the various cannabinoids found in the processed Cannabis-Hemp Plant.

Then after applied for the ‘Cannabis-Hemp Dip’ patent (Ser. No. 15/084,336) and the ‘Cannabis-Hemp Cigarette’ patent (Ser. No. 15/082,278), my dear friend contracted heart disease. So I quickly started studying the effects of red wine on heart disease, then added resveratrol to my ‘Cannabis-Hemp Paste’, and then got her to take eight enhanced cannabis-hemp capsules a day (paste), while also going mostly organic, and mostly vegetarian. Her shortness of breath, and her heart pains, quickly dissipated.

And as a result of this singular success, then asked myself how we could infuse the cannabinoids with everyday non-alcoholic beverages, as well as with beer and wine, while using my previously discovered cannabis-hemp processing methods. And that's when came up with the Cannabis-Hemp Beverage Infusions patent application, which you are reading now.

### SUMMARY

The ‘Cannabis-Hemp Beverage Infusions’ process is a novel, non-obvious and useful method to transform the Cannabis-Hemp Plant into a pleasant and drinkable infusion.
A primary object of the present invention is to provide an inexpensive method to commercially process the Cannabis-Hemp plant into a soothing, impactful, and pleasant beverage, so that the process overcomes the shortcomings of the other methods.
Another object is to provide Cannabis-Hemp Beverage Infusions that utilize the entourage effect, while reducing the amount of wasted cannabinoids, terpenes, terpenoids, and flavonoids that are being thrown away after current extraction methods.
Another object is to provide Cannabis-Hemp Beverage Infusions that utilize the entourage effect, while reducing the amount of cannabinoids being wasted through only a single heat cooking step (therefore not making many of the cannabinoids bioavailable for infusion).
An additional object is to provide Cannabis-Hemp Beverage Infusion that can be stored for extended periods of time, while processing the infusions into various flavor profiles, so as to create a soothing, impactful, and pleasant beverage.
Another object is to provide Cannabis-Hemp Beverage Infusions that utilize the entourage effect, by including properly stored raw cannabis-hemp flowers to the final infusion, so as to provide the full complement of wholesome cannabinoids, terpenes, terpenoids, and flavonoids.
Another object to provide affordable Cannabis-Hemp Beverage infusions that can be used regularly, so as to enhance soothing and recreational beverages.
   *Further objects of the invention will appear as the description proceeds.

Briefly described, in its preferred form, the present invention provides a method for preparing a commercially inexpensive way to produce a high quality Cannabis-Hemp Beverage infusion. The present process preferably comprises the following steps:
a. preparing the infusion by heat-cooking the raw flowers for decarboxylation
b. adding various infusions, fillers, chemicals and flavorings to the cooked cannabis-hemp flowers
c. preparing the infusion by heat-cooking the enhanced cannabis-hemp flowers for the second time
d. adding various infusions, herbs, fillers, chemicals and flavorings to the enhanced and twice-heat-cooked cannabis-hemp flowers
e. preparing the infusion by blending the raw cannabis-hemp flowers, to the twice cooked flowers.
f. adding raw cannabis-hemp flower to the enhanced ‘cannabis-hemp flower mix’ before steeping
g. allowing time for the raw and twice-decarboxylated enhanced cannabis-hemp flower mix to steep
h. begin final processing by straining certain-sized particulate matter from the cannabis-hemp infusion
i. process the final cannabis hemp fusionable beverage into sealed drinking containers

Cannabis-Hemp Beverage Infusions are best when the Cannabis-Hemp is freshly harvested and used for infusions. That is, the most desirable Cannabis-Hemp Beverage infusions begin with a freshly harvested product in the beginning of the process. While a fresh initial product is preferred, the present process can accommodate a cannabis-hemp flower (including leaves and stems) that has been frozen and/or dried and cured.

The heat-cooking temperature, and the time period the product stays within the cooking chamber, can be adjusted for the various decarboxylation processes, that the present invention is designed to handle. Each component step of the present invention can similarly be adjusted. Some of the adjustments can be incorporated by simply manipulating heat duration, and heat temperatures.

The preferable heat-cooking steps may comprise only one dry heat cooking step, multiple dry heat-cooking steps, and/or dry heat-cooking step(s) with blended herbs, chemicals, and flavorings. Yet if the product is not twice-cooked with heat (varying times and temperatures), the final product lacks the amount of decarboxylated cannabinoids of the twice-heat-cooked product. And If the final product uses only raw cannabis-hemp flowers (with no heat cooking), then the cannabinoids won't be made bioavailable, and the process will produce an inferior product.

In the conventional processing methods, the process incorporates cannabinoid extractions, and/or the use of one heat-cooking step. In addition, the conventional processing methods typically use improperly stored Cannabis-Hemp Flowers, which are often contaminated with mold. As a result of the conventional processing methods, the consumer won't realize the full efficacy of the decarboxylated cannabinoids.

In the present process, the herbs, chemicals and flavoring are added after the initial convection-cooking step, and a processed portion of the final infusion is never heated so as to preserve the naturally occurring terpenes, terpenoids, and flavonoids. Thus, the product retains its full compliment of healthy ingredients because differently processed plant materials are added throughout the various blending steps; to create both the enhanced, and superior product, known as the ‘Cannabis-Hemp Beverage Infusions’.

If the fusionable beverage is too hot at any point in the process, then many of the cannabinoids will be lost. If the fusionable beverage is too cool during the heat-cooking process, then the cannabinoids won't decarboxylate and the product won't be as effective.

Unlike the conventional processes, the present invention combines both processed and cooked aspects of the cannabis-hemp plant, to the final Cannabis-Hemp Beverage Infusions. Thus upon ingesting, the biochemical affects can help upregulate the endocannabinoid immune system, which then helps provide a biochemical homeostasis, which can then help us self-heal through a daily healthful regimen.

The present invention enables a single location to process, cure, store, freeze, refrigerate, cook and package the ‘Cannabis-Hemp Beverage Infusions’ in large quantities. Therefore, each retail outlet can eliminate any reliance upon, and the capital expense for, third party quality certifications. They need simply to refer them to the publicly published materials, and the transparent process, being shared by the current patent application owner.

Finally, the novel and useful ‘Cannabis-Hemp Beverage Infusion’ process, utilizes several steps in the production of the final ‘Cannabis-Hemp Beverage Infusions’. Accordingly, an object of the invention is to provide a commercial process for preparing of an affordable Cannabis-Hemp Beverage Infusions that can be used regularly, and with no harmful side effects.

### DETAILED DESCRIPTION

The present invention provides a commercial process for preparing Cannabis-Hemp Beverage Infusions. The process produces both a processed, heat-cooked, and enhanced Cannabis-Hemp Beverage Infusion that can be processed and poured into a drinking vessel of any type.

To process the ‘Cannabis-Hemp Beverage Infusions’, the steps include:
providing a quantity of fresh or properly stored raw cannabis-hemp (flowers, stems, and leaves); providing a means to convey the raw cannabis-hemp product through the various steps of the present invention; processing the cannabis-hemp plant; processing other herbs and blending sources and fillers to be added later; heat cooking a portion of the processed flowers; adding chemicals and flavorings to the once heat-cooked flowers; re-heat cooking the previously heat-cooked flowers with the added chemical and flavorings; blending the correct ratio of dried leaves, processed flowers, and heat-cooked flowers; with the other blending sources and fillers; steeping the final product into various beverage infusions; packaging the final product for proper storage; and labeling the product for education purposes.

1. A process for preparing a beverage with cannabis-hemp infusions, comprising the ordered steps of:
a) providing a quantity of the freshly harvested cannabis-hemp pant (the initial ‘fusionable product’)
b) providing a system to convey the ‘fusionable product’ through the various steps of the process
c) preparing other ‘fusionable products’ for later additions to the fusionable beverage
d) preparing the raw cannabis-hemp infusion by removing the seeds from the stalks
e) preparing the raw cannabis-hemp infusion by drying and curing a portion of the cannabis-hemp flowers, and/or by placing a portion of the cannabis-hemp flowers in cold storage (freezer or refrigeration).
f) preparing the fusionable cannabis-hemp flowers by heat cooking the fresh or stored cannabis-hemp flowers for decarboxylation; so as to make many of the cannabinoids bioavailable
g) adding the other prepared fusionable products (a portion of other herbs, fruits, chemicals, and flavorings) and the heat cooked flower to the ‘cannabis-hemp flower mix’.
h) heat-cooking the infusion, including the enhanced and once-decarboxylated cannabis-hemp flower mix (now being heat cooked for the second time), thus creating an amalgam of decarboxylated cannabinoids for the steeping process.
i) blending the proper ratios of the enhanced ‘cannabis-hemp flower mix’ to the fruit infusion
j) adding raw cannabis-hemp flower and fresh fruit to the ‘cannabis-hemp flower mix’ before steeping
k) allowing time for the raw and twice-decarboxylated enhanced cannabis-hemp flower mix to steep
l) begin final processing by straining certain-sized particulate matter from the cannabis-hemp infusion
m) process the final cannabis hemp fusionable beverage into sealed drinking containers while adding the final flavor and preservative enhancements.

2. The process of preparing the ‘cannabis-hemp fusionable beverage’ according to claim 1, wherein the fusionable beverage is fully prepared upon exiting the steps of twice-heat-cooking the flowers (h); without all, each, or a combination of the additional fruits, herbs, fillers, chemicals and flavorings (c through j).

3. The process of preparing the ‘cannabis-hemp fusionable beverage’ according to claim 1, wherein the fusionable beverage is fully prepared upon exiting the steps of twice-heat cooking the flowers (h); without the steps of properly storing the cannabis-hemp product (d and e).

4. The process of preparing the ‘cannabis-hemp beverage infusions’ according to claim 1, wherein the fusionable beverage is fully prepared upon exiting the steps of twice-heat-cooking the flowers (h) and wherein the fusionable beverage is fully prepared after blending the twice-heat-cooked and raw cannabis-hemp fusionable beverage (j) together; without all, each, or a combination of the additional herbs, fruits, fillers, chemicals, and flavorings.

5. The process of preparing ‘cannabis-hemp beverage infusions’ according to claim 1, comprising the further step of combining the fusionable beverage with various chemicals, spices, salts, seeds, nuts, roots, flavorings, colorings, condiments, fruits, fruit peels, essential oils, herbs, flowers, leaves, vegetables, extracts, and sweeteners before and after steps (c through m).

6. The process of preparing ‘cannabis-hemp beverage infusions’ according to claim 1, comprising the further step of evacuating the sealed curing jugs, so as to help prevent the formation of any fungal matter.

7. The process of preparing ‘cannabis-hemp beverage infusions’ according to claim 1, removing the step of adding any fruit(s), herb(s), chemical(s), flavoring(s), and preservatives to the infusion.

8. A thrice-cooked process for preparing the ‘fusionable beverage’ according to claim 1, comprising the steps of:
a. adding various herbs, fillers, chemicals and flavorings to the dried and cured flowers
b. preparing the fusionable beverage by heat-cooking the enhanced flowers for decarboxylation
c. adding various fruits, herbs, fillers, chemicals and flavorings to the enhanced heat-cooked flowers
d. re-heat-cooking the enhanced cannabis-hemp flowers to further decarboxylate the cannabinoids
e. preparing the fusionable beverage by blending the dried cannabis-hemp leaves, to twice cooked flowers.
f. blending fruits, herbs, chemicals, and flavorings, to the twice heat-cooked cannabis-hemp blend
g. heat cooking the enhanced twice cooked cannabis-hemp blend for the third time
h. allowing time for the raw and thrice-decarboxylated enhanced cannabis-hemp flower mix to steep with fruit infusion
i. begin final processing by straining certain-sized particulate matter from the cannabis-hemp infusion
j. process the final cannabis-hemp beverage infusion into sealed drinking containers, while adding the final flavor and preservative enhancements.

9. The process of preparing the ‘cannabis-hemp beverage infusions’ according to claims 1 through 8, comprising the further step of freezing and or refrigerating the fusionable beverage before and after a step, a combination of steps, or every step (a to m).

10. The process of preparing the ‘cannabis-hemp beverage infusions’ according to claims 1 through 9, comprising the further step of fermenting the cannabis-hemp beverage infusions before and after a step, a combination of steps, or every step (a to m).
